(19) United States
(12) Patent Application Publication
(10) Pub. No.: US 2023/0389996 A1
(43) Pub. Date: Dec. 7, 2023

(54) MEDICAL SYSTEMS, DEVICES, AND RELATED METHODS

(71) Applicant: Boston Scientific Scimed, Inc., Maple Grove, MN (US)

(72) Inventors: Daragh NOLAN, Youghal (IE); Thomas SINNOTT, Enniscorthy (IE); Garret REYNOLDS, Dublin (IE); Daniel J. FOSTER, Lino Lakes, MN (US); Linda MAHER, Tullaroan (IE); Kyle TRUE, Minneapolis, MN (US); Sebastian ORDAS CARBONI, Vadnais Heights, MN (US); Andrew Brian GRAVELEY, Shoreview, MN (US)

(73) Assignee: Boston Scientific Scimed, Inc., Maple Grove, MN (US)

(21) Appl. No.: 18/330,088

(22) Filed: Jun. 6, 2023

Related U.S. Application Data
(60) Provisional application No. 63/349,608, filed on Jun. 7, 2022.

(51) Int. Cl. A61B 34/20; A61B 1/018; A61B 1/015

(52) U.S. Cl. CPC A61B 34/20; A61B 1/018; A61B 1/015; A61B 1/307

(57) ABSTRACT

Medical systems are described, including a medical system includes a first medical device and a second medical device. The first medical device includes a handle and an insertion portion with a first working channel. A distal portion of the insertion portion of the first medical device includes a first tracking element and at least one camera. The second medical device includes a handle and an insertion portion. A distal portion of the insertion portion of the second medical device includes a second tracking element. The first tracking element is configured to provide a signal that indicates a position or orientation of the distal portion of the first medical device. The second tracking element is configured to provide a signal that indicates a position or orientation of the distal portion of the second medical device.

### CROSS-REFERENCE TO RELATED APPLICATIONS

This application claims the benefit of priority to U.S. Provisional Application No. 63/349,608, filed on Jun. 7, 2022, which is incorporated by reference herein in its entirety.

### TECHNICAL FIELD

The disclosure relates generally to medical systems, devices, and related methods. More specifically, aspects of the disclosure pertain to medical systems, devices, and related methods including medical devices with position-tracking and/or mapping assemblies.

### BACKGROUND

In a medical procedure, an operator may insert a medical device, such as a ureteroscope or other type of scope, into a body lumen of a subject. The operator may navigate a distal tip of the medical device to a desired location of the subject's anatomy, for example, through the subject's urethra, bladder, ureter, and/or kidney. Prior to the medical procedure, an area of interest in the subject's anatomy may be predefined. The operator may then attempt to navigate the medical device to that predefined area of interest. Such navigation may be challenging, particularly in tortuous passages or complicated areas of anatomy (e.g., urethra, bladder, ureter, kidneys, etc.). An imager (e.g., a camera) at a distal tip of the medical device may facilitate such navigation, but cannot provide information about areas past the walls of the body lumen. Effective navigation using only an imager may require high levels of skill, time, and/or effort. Additionally, navigation with only an imager may not allow the user to determine a position and/or orientation of the distal tip of the medical device, for example, relative to one or more areas of anatomy and/or objects (i.e., kidney stones) within the subject. Navigation may be aided by one or more external imaging techniques, but such external imaging techniques may increase the cost, duration, risks, etc. of the procedure.

An endoscopic robotic system may rely on position information for a distal tip of a medical device. Some robotic systems utilize position encoders in a motor system of the robotic system. Such sensors, however, do not provide useful information for a flexible tool (such as a ureteroscope, other type of scope, a biopsy catheter, biopsy tool for passing through a working channel of an endoscope, or another type of flexible tool). Therefore, a need exists for systems, devices, and/or methods for including position-tracking and/or mapping assemblies.

### SUMMARY

A medical system may include a first medical device and a second medical device. The first medical device may include a handle and an insertion portion with a first working channel. A distal portion of the insertion portion of the first medical device may include a first tracking element and at least one camera. The second medical device may include a handle and an insertion portion. A distal portion of the insertion portion of the second medical device may include a second tracking element. The first tracking element may be configured to provide a signal that indicates a position or orientation of the distal portion of the first medical device. The second tracking element may be configured to provide a signal that indicates a position or orientation of the distal portion of the second medical device.

The medical system may include one or more of the following aspects. The distal portion of the first medical device further may include one or more lighting elements. The first tracking element, the at least one camera, and the one or more lighting elements may be coupled to a substrate within the distal portion of the first medical device. The substrate may include a first face and a second face. The first face and the second face may be arranged approximately orthogonally to each other within the distal portion of the first medical device. A proximal portion of the substrate may include a plurality of contact pads.

The first tracking element may include a position sensing system that includes one or more position or orientation sensors. The position sensing system may include a first magneto-resistive sensor, a second magneto-resistive sensor, and a third magneto-resistive sensor. The first magneto-resistive sensor, the second magneto-resistive sensor, and the third magneto-resistive sensor may be coupled to the substrate. The first magneto-resistive sensor may have a first primary sensing direction. The second magneto-resistive sensor may have a second primary sensing direction. The third magneto-resistive sensor may have a third primary sensing direction. The first magneto-resistive sensor and the second magneto-resistive sensor may be arranged so that each of the first primary sensing direction and the second primary sensing direction is approximately parallel with a longitudinal axis of the substrate. The third magneto-resistive sensor may be arranged such that the third primary sensing direction is transverse to the longitudinal axis of the substrate. The position sensing system may be configured to measure at least five degrees of freedom. The distal portion of the first medical device further may include one or more diodes and one or more capacitors. The one or more diodes and the one or more capacitors may be coupled to the substrate.

The handle of the first medical device may include a lever configured to control an articulation or deflection of the distal portion of the first medical device. The handle of the first medical device may include a port connected to the first working channel. The handle of the first medical device may include a suction valve to control suction applied through the first working channel. The first medical device may include an optical fiber extending from the handle portion to the distal portion of the insertion portion of the first medical device. A distal end of the optical fiber may be adjacent to the at least one camera. The first medical device further may include a pressure sensor positioned at the distal portion of the first medical device. The second medical device may be a nephroscope. The nephroscope may include a working channel. The medical system may further include a retrieval device configured to be delivered through the working channel of the nephroscope. The first medical device may include an end cap at the distal portion. The end cap may at least partially surround the first tracking element.

In another aspect, a medical device may include a handle and an insertion portion. The handle may include a port. The insertion portion may include a working channel connected to the port. A distal portion of the insertion portion may include a tracking element and at least one camera. The tracking element may include a plurality of magneto-resistive sensors. The tracking element may be configured to provide a signal that indicates a position or orientation of the distal portion of the medical device. The tracking element and the at least one camera may be mounted on a substrate within a distal portion of the insertion portion.

The medical device may include one or more of the following aspects. The substrate may include a first face and a second face. The first face and the second face may be arranged approximately orthogonally to each other within the distal portion of the insertion portion of the medical device. The at least one camera may be mounted on the first face of the substrate. The plurality of magneto-resistive sensors may include a first magneto-resistive sensor, a second magneto-resistive sensor, and a third magneto-resistive sensor. The first magneto-resistive sensor, the second magneto-resistive sensor, and the third magneto-resistive sensor may be coupled to the second face of the substrate. The handle may further include an image capture button, a lever, a port, and a suction valve. The image capture button may be configured to control activation of the camera at the distal portion of the insertion portion. The lever may be configured to control an articulation or deflection of the distal portion of the medical device. The suction valve may be configured to control suction applied through the working channel.

In yet another aspect, a method may include delivering a first medical device into a subject. The first medical device may include a handle and an insertion portion. The insertion portion may include a working channel. A distal portion of the insertion portion may include a camera and a tracking element. The tracking element may include one or more magneto-resistive sensors. The method may also include delivering the distal portion of the insertion portion the first medical device into a body lumen or body cavity of the subject. The method may include identifying and/or mapping one or more objects within the body lumen or the body cavity with the camera. Furthermore, the method may include determining whether the one or more objects can be removed from the body lumen or body cavity with the working channel of the first medical device. The method may include, upon determining that the one or more objects cannot be removed with the working channel of the first medical device, providing a proposed access path for a second medical device to the body lumen or body cavity of the subject. The method may include aiming and inserting the second medical device to the body lumen or the body cavity of the subject. The method may also include tracking a position of the second medical device within the subject. Moreover, the method may include removing or otherwise treating the one or more objects within the body lumen or body cavity with the second medical device.

The method may include one or more of the following aspects. The method may include an initial step of preparing the subject for a procedure by performing one or more of an ultrasound, a CT scan, or an MRI scan of a kidney of the subject. The method may include, before providing the access path for the second medical device, overlaying one or more overlaying one or more aspects of the kidney or one or more kidney stones on the subject.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts an exemplary medical system, including first and second medical devices.

FIGS. 2A-2D are side, perspective, and cross-sectional views of a distal portion of the first medical device, including a distal component assembly.

FIGS. 3A-3E depict various views of the distal component assembly of FIGS. 2A-2D.

FIG. 4 is a flow diagram of an exemplary method, according to aspects of this disclosure.

### DETAILED DESCRIPTION

Robotic-assisted and electromagnetic (“EM”)-navigated medical procedures may utilize EM tracking to provide information regarding a position and/or orientation of a medical device within a subject's anatomy. When a medical device has a camera, an operator is unable to see past an endothelial wall, or other type of wall or body structure. With EM tracking, position and/or orientation information may be fused with imaging (e.g., three-dimensional (“3D”) imaging) performed before a procedure and/or imaging received from the camera. An operator may have greater information about the anatomy near the medical device(s), which a camera alone may be unable to visualize (including anatomy outside of a body lumen in which the medical device is disposed). Furthermore, pre-procedure images and/or imaging from a camera at the distal tip of the medical device(s) may be used to automatically segment a mesh of the anatomy so as to provide a map (e.g., a 3D map) to track the medical device(s) in real time, for example, to build a 3D representation of the anatomy (e.g., a 3D map) to help navigate the medical device(s) within the anatomy. Such real-time tracking and/or guidance may help to decrease the amount of time, skill, and/or effort required to reach a target anatomy. In the absence of pre-procedure images, EM sensors may enable one or more controllers (i.e., including stored software) to track the position, orientation, and/or movements of the medical device(s) in order to generate a map (e.g., a 3D map) in real time, during a procedure. Additionally, EM sensors may help to accelerate the generation of the map (e.g., the 3D map). The generated map may guide the medical device(s) (and any EM-enabled accessories and/or any EM-tracked accessories) through the subject's anatomy. Unlike traditional robotic systems, which may rely on position encoders in a motor system to provide necessary position information for a tip of a medical device, such as a rigid medical device or a rigid portion of a medical device, EM-based systems may provide position information for flexible medical devices.

Additionally, in some aspects, a single circuit board at a distal end of a medical device may include elements such as position-sensing systems, imaging elements, and lighting elements. The position-sensing elements may enable EM tracking of the medical device. For example, a position-sensing system may include one or more tunneling magnetoresistance (“TMR”) sensors (i.e., TMR elements) or other magnetoresistive (“MR”) sensors (i.e., MR elements), one or more diodes (e.g., two diodes), and/or one or more capacitors (e.g., one capacitor). Imaging elements may include one or more cameras. Lighting elements may include one or more light emitting diodes (“LEDs”) and/or fiber optic light guides. Wires, cables, or other conductors for carrying power and/or signals to the elements of the circuit board may extend proximally from the circuit board, toward a proximal end of the medical device(s). Various elements may be arranged on the circuit board to arrange for efficient connections between the circuit board and the conductors carrying power and/or signals.

A distal portion (e.g., a housing or end cap) of the medical device may be configured to receive the circuit board. Inclusion of position sensing elements, imaging elements, and/or lighting elements on a single circuit board may facilitate cost-effective manufacturing by, for example, reducing a number of steps to assemble the medical device, reducing a likelihood of errors in assembly, and/or reducing waste. For example, rather than separately assembling lighting elements, imaging elements, and/or sensing elements on or within a distal tip body, all of these elements (and the conductors providing power and/or signals thereto) may be fitted to the distal tip body in a single step.

Examples of this disclosure include systems, devices, and methods for facilitating and/or improving the efficacy, efficiency, cost, and/or safety of a medical procedure. Embodiments of the disclosure may relate to systems, device, and methods for performing various medical procedures and/or treating portions of the kidneys, ureters, bladder, urethra, or any other portion of the urinary tract. Additionally, embodiments of the disclosure may relate to systems, devices, and methods for performing various medical procedures and/or treating portions of the large intestine (colon), small intestine, cecum, esophagus, stomach, or any other portion of the gastrointestinal tract or biliary tree. Furthermore, embodiments of the disclosure may relate to systems, devices, and methods for performing various medical procedures and/or treating portions of anatomy, for example, accessed via a body lumen, such as the larynx, trachea, bronchi (primary bronchi), lobar (secondary bronchi), segmental (tertiary bronchi), or any other portion of the respiratory system. In these aspects, the systems, devices, and methods discussed herein may be used to treat any other suitable subject anatomy (collectively referred to herein as a “treatment site”).

Various embodiments described herein include single-use or disposable medical devices. Some aspects of the disclosure may be used in performing an endoscopic, arthroscopic, bronchoscopic, ureteroscopic, colonoscopic, or other type of procedure. Some aspects of the disclosure may be used in performing a neuromodulation procedure, for example, to help locate one or more electrodes used in a therapy or procedure (e.g., in deep brain stimulation). For example, the disclosed aspects may be used with ureteroscopes, endoscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, catheters, diagnostic or therapeutic tools or devices, or any other suitable delivery device or medical device, for example, for treatment through a body lumen. Alternatively, various embodiments described herein may be delivered to a treatment site alone and/or used separate from another scope or medical device. One or more of the elements discussed herein could be metallic, plastic, or include a shape memory metal (such as Nitinol), a shape memory polymer, a polymer, or any combination of biocompatible materials.

Reference will now be made in detail to examples of the disclosure described above and illustrated in the accompanying drawings. Wherever possible, the same reference numbers will be used throughout the drawings to refer to the same or like parts. It is noted that one or more aspects of the medical systems, devices, and methods discussed herein may be combined and/or used with one or more aspects of other medical systems, devices, and methods discussed herein.

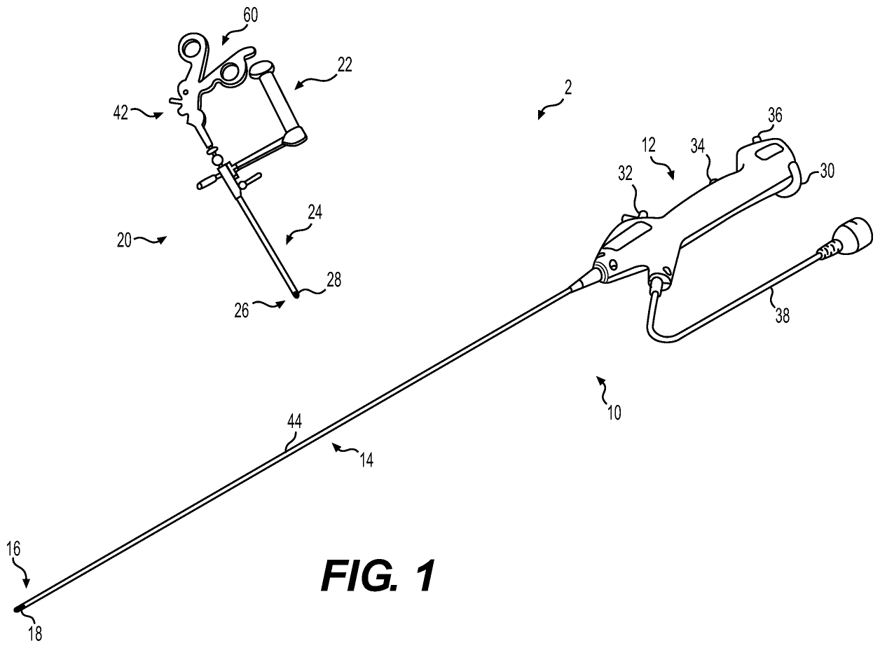

FIG. 1 depicts aspects of an exemplary medical system 2. Medical system 2 includes a first medical device 10 and a second medical device 20. First medical device 10 may include a handle portion 12 for gripping and operation by an operator, and an insertion portion 14 for at least partial insertion into a body (e.g., a body lumen) of a subject. A distal portion 16 of insertion portion 14 may include one or more tracking elements 18 (e.g., an EM-based sensor). As shown in FIG. 1, first medical device 10 may include a ureteroscope. Although the disclosure may refer at different points to a ureteroscope or an endoscope, it will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the elements and assemblies described herein. Second medical device 20 may be a nephroscope, which may include a handle portion 22 and an insertion portion 24. A distal end 26 of insertion portion 24 may include one or more tracking elements 28 (e.g., an EM-based sensor). Additionally, second medical device 20 may receive one or more retrieval devices 42. One of more of first medical device 10 and/or second medical device 20 may be used to track and/or map the position of respective distal portions of insertions portions 14, 24, map one or more internal cavities of a subject, locate one or more objects or tissues to be removed from the one or more internal cavities of the subject, and/or remove one or more objects or tissues from the one or more internal cavities of the subject.

Handle portion 12 of first medical device 10 may include a lever 30, for example, on a proximal portion of handle portion 12. Lever 30 may help to facilitate articulation, steering, deflection, or other movement of insertion portion 14, including distal portion 16. Although lever 30 is depicted in FIG. 1, it will be appreciated that any suitable actuator(s) may be used in addition to or in place of lever 30, such as one or more knobs, buttons, sliders, or joysticks.

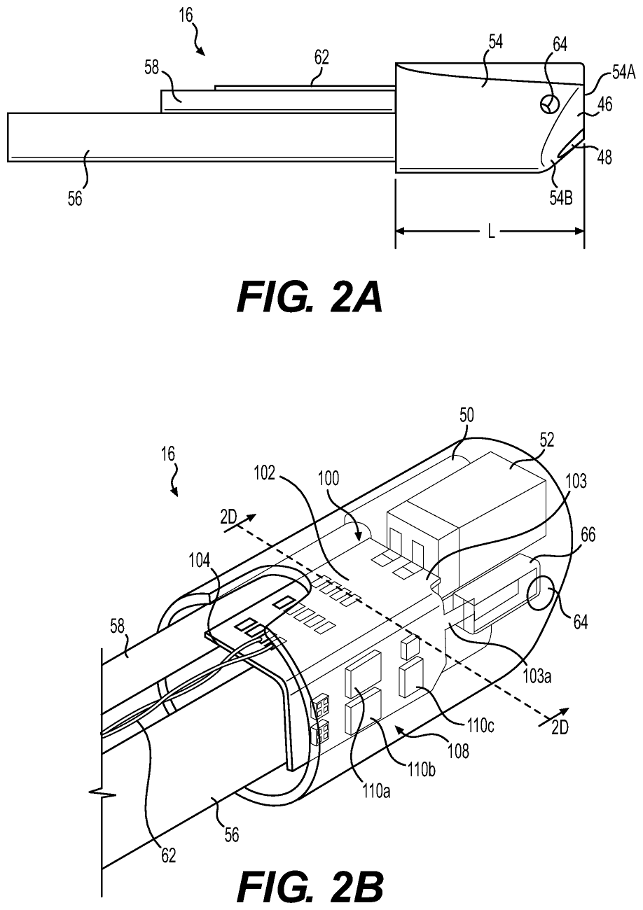

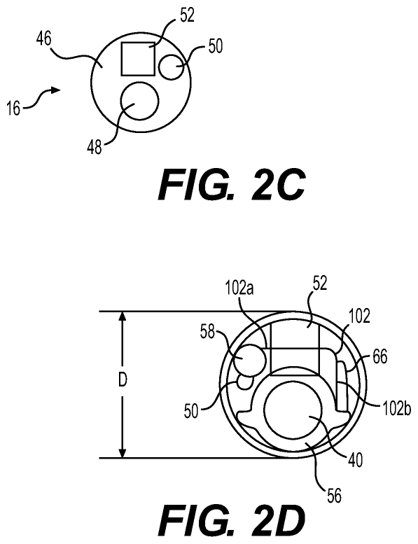

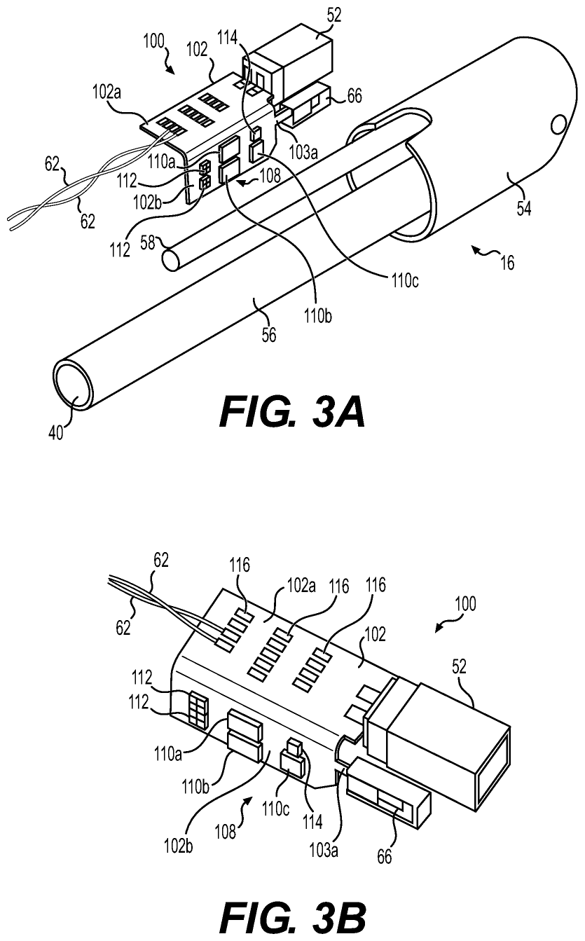

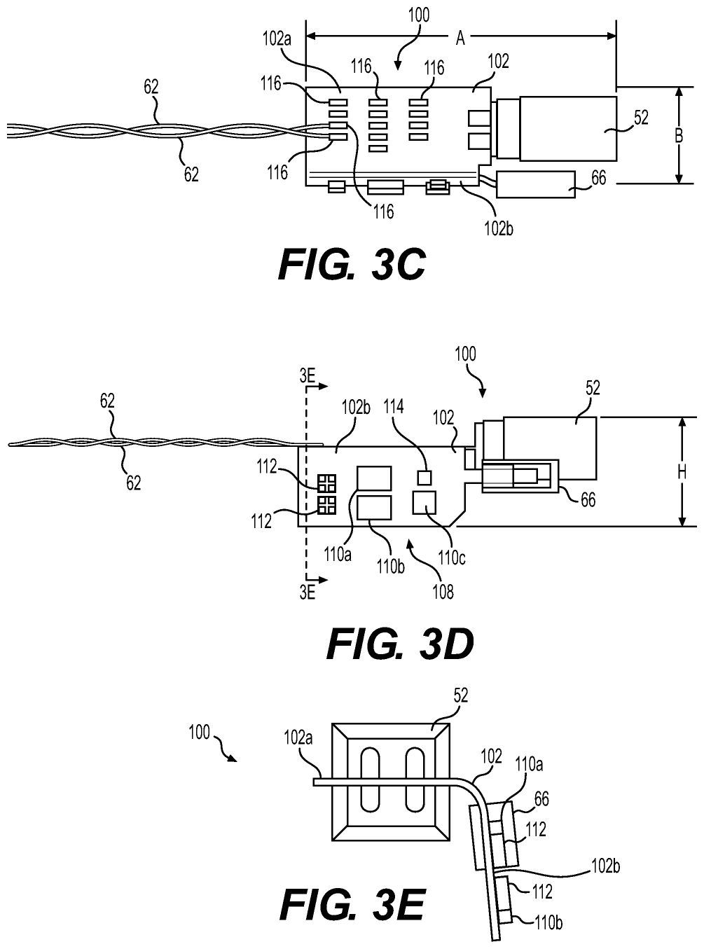

A port 32 of handle portion 12 (e.g., on a proximal portion of handle portion 12) may provide access to a lumen or working channel 40 (FIGS. 2D and 3A) of first medical device 10. In some aspects, port 32 may be a T-shape port, and/or may include one or more valves, luers, etc. An operator may insert an instrument or other device into port 32 and may extend the instrument or other device distally through working channel 40. Working channel 40 may extend longitudinally through a length of insertion portion 14.

Handle portion 12 may also include a suction valve 34, for example, on a proximal portion of handle portion 12 and on an opposing side from lever 32. An operator may connect suction valve 34 to a source of suction, and may operate suction valve 34 to generate suction through insertion portion 14 (e.g., through working channel 40). Handle portion 12 may additionally or alternatively include other types of valves, such as air and/or water valves, or valves that perform a combination of functions.

Handle portion 12 may also include an image capture button 36. Image capture button 36 may enable an operator to capture a still image from a camera 52 (shown in FIGS. 2B and 2C and described in further detail below) during a procedure. Image capture button 36 may be positioned on a proximal portion of handle portion 12, for example, adjacent suction valve 34. Additionally or alternatively, image capture button 36 may enable an operator to capture video or to perform other functions to control first medical device 10.

Handle portion 12 of first medical device 10 may be coupled to an umbilicus 38. Umbilicus 38 may extend from handle portion 12 (e.g., from a distal portion of handle portion 12) and may carry wires, cables, and/or conduits for providing, for example, power, signals, or fluids to or from handle portion 12. For example, umbilicus 38 may connect handle portion 12 to one or more user interfaces, monitors, displays, fluid sources, fluid storage containers (i.e., removed from a subject via suction), etc.

Insertion portion 14 of first medical device 10 may include a shaft 44 extending distally from handle portion 12. Shaft 44 may have any suitable properties. For example, shaft 44 may be flexible and may have wires, tubes, or other features passing therethrough. Distal portion 16 of first medical device 10 may be disposed at a distal end of shaft 44. As shown in FIGS. 2A and 2C, distal portion 16 may include a distalmost face 46. Distalmost face 46 may define a working channel opening 48. Working channel 40 may extend between port 32 and working channel opening 48, such that instruments or other devices may be passed through port 24, through working channel 40, and out of working channel opening 48. An instrument extending distally of working channel opening 48 may be used to perform a medical procedure on a subject.

Distal portion 16 may also include imaging components, such as one or more illumination devices or lighting elements 50 and one or more imaging devices or cameras 52 (FIGS. 2B-2D). Although one lighting element 50 and one camera 52 are depicted in FIGS. 2B-2D, it will be appreciated that alternative numbers of lighting elements 50 and camera 52 may be utilized. Alternatively, lighting element(s) 50 and camera 52 may be combined into a single device. Lighting element(s) 50 may include LEDs, optical fiber(s), or any suitable alternative light source(s). Camera 52 may be configured to capture or otherwise take video and/or still images. Camera 52 may provide a signal to a monitor (not shown), e.g., via one or more cables or wires through shaft 44 and/or umbilicus 38, so that an operator may view a visual image provided by camera 52 while navigating and/or manipulating first medical device 10 through a body lumen or body cavity of a subject. Additionally, a control unit or controller (not shown), either integral with first medical device 10 or separate from first medical device 10 may analyze one or more image and/or video signals, for example, to create a map (e.g., a three-dimensional map) of one or more body lumens or body cavities within the subject.

As depicted in FIGS. 2A and 2C and described above, first medical device 10 may be “forward-facing.” In other words, features of distal portion 16 (e.g., working channel opening 48, lighting elements 50, and camera 52) may face distally (i.e., forward of distalmost face 46). This disclosure also encompasses other configurations of distal portion 16. For example, first medical device 10 may be “side-facing.” In a side-facing embodiment, one or more of working channel opening 48, lighting elements 50, and/or camera 52 may be disposed on a radially outer side of distal portion 16. In these aspects, one or more of working channel opening 48, lighting elements 50, and/or camera 52 may point or face in a radially outward direction, approximately perpendicularly or otherwise transversely to a longitudinal axis of insertion portion 14 of first medical device 10.

As mentioned, second medical device 20 may be a nephroscope, for example, including handle portion 22 and insertion portion 24. Handle portion 22 may receive one or more instruments, for example, retrieval device 42. Retrieval device 42 may include one or more actuation members 60, for example, movable to open and/or close an end effector (e.g., a grasper, an expandable basket, etc.) that may be extended through second medical device 20 and extend distally of distal portion 26 of insertion portion 24. Furthermore, distal portion 26 may include tracking element 28, as mentioned above. Additionally, although not shown, one or more portions of second medical device 20 may be delivered to a lumen of a subject via an insertion device. For example, a portion of insertion portion 24 may be delivered to a subject's kidney via one or more of a guidewire, a tubular member, or a sheath, for example, for a percutaneous nephrolithotomy (“PCNL”) procedure.

FIGS. 2A-2D depict different views of distal portion 16 of insertion portion 14 of first medical device 10. FIG. 2A is a side view of distal portion 16 of insertion portion 14, and FIG. 2B is a perspective view of distal portion 16 of insertion portion 14 (i.e., looking distally from a position proximal of distal portion 16). FIGS. 2A and 2B illustrate portions of insertion portion 14 with an outer sheath or covering (FIG. 1) removed.

As shown in FIG. 2A, insertion portion 14 may include an end cap 54, for example, at a distal end of the insertion portion 14. End cap 54 may be coupled to a distal end of insertion portion 14, or may be integrally formed with insertion portion 14. In some aspects, end cap 54 may form distalmost face 46. Furthermore, in some aspects, end cap 54 may include a flat distal face 54A and an angled or tapered face 54B, for example, extending proximally at an angle from flat distal face 54A. In some aspects, end cap 54 may include a longitudinal length L of approximately 0.2 inches to approximately 0.3 inches, for example, approximately 0.25 inches, approximately 0.21 inches, etc. End cap 54 may at least partially surround one or more portions of tracking element 18.

In these aspects, as shown in FIGS. 2A and 2B, insertion portion 14 may include a working channel lumen 56 and an optical fiber 58. As mentioned, working channel lumen 56 may form working channel 40, and may connect port 32 to working channel opening 48. Optical fiber 58 may extend approximately parallel to at least a portion of working channel lumen 56, for example, extending from handle portion 12 to (or just proximal to) distalmost face 46. Optical fiber 58 may form or be coupled to lighting element(s) 50.

Furthermore, insertion portion 14 may include one or more cables or wires 62, for example, to electrically and/or communicatively couple one or more portions of distal portion 16 to handle portion 12, umbilicus 38, etc. As shown in FIGS. 2A and 2B, wires 62 may be twisted, for example, to form a bundle. Moreover, as shown in FIGS. 2A and 2B, end cap 54 may include one or more coupling elements 64 (e.g., screws, bolts, etc.), for example, to couple portions of end cap 54 together and/or to allow for portions of end cap 54 to be uncoupled to provide access to the interior of end cap 54. Alternatively, portions of end cap 54 may be coupled with an adhesive, a snap-fit, etc.

As shown in FIG. 2B and as discussed in greater detail below, distal end 16 may include a distal component assembly 100, which may constitute or be included in a medical assembly. As discussed in further detail below, with reference to FIGS. 3A-3E, distal component assembly 100 may be disposed in distal portion 16 of first medical device 10, for example, within end cap 54.

Distal component assembly 100 may include a substrate 102 (e.g., a circuit board or other type of board). Substrate 102 may include, for example, a rigid or flexible printed circuit board and may include one or more layers. In one example, substrate 102 is flexible and includes multiple layers. A proximal end 103 of distal component assembly 100 may be a leftmost side in FIG. 2B. A distal end 104 of distal component assembly 100 may be a rightmost side in FIG. 2B. One or more of lighting elements 50 and/or camera 52 may be supported by or otherwise coupled to substrate 102. Additionally, a pressure sensor 66 may be supported by or otherwise coupled to substrate 102, for example, via a distal extension 103a.

As discussed in greater detail below, one or more elements of a position sensing system 108 may also be disposed on substrate 102. Position sensing system 108 may incorporate any of the features described in U.S. application Ser. No. 15/846,846, filed on Dec. 19, 2017, issued as U.S. Pat. No. 10,782,114, on Sep. 22, 2020, the entirety of which is incorporated herein by reference.

Position sensing system 108 may include one or more magnetic field sensors 110a, 110b, 110c disposed on substrate 102. For example, as shown in FIG. 2B, three magnetic field sensors 110a, 110b, 110c may be disposed on substrate 102, and may form tracking element 18. Any alternative number of sensors may be utilized, and the three sensors 110a, 110b, 110c depicted are exemplary only. Magnetic field sensors 110a, 110b, 110c may include, for example, MR elements, such as TMR elements, anisotropic-magneto-resistive sensing elements, giant magneto-resistive sensing elements, colossal magneto-resistive sensing elements, extraordinary magneto-resistive sensing elements, or semiconductor magneto-resistive elements. Additionally or alternatively, magnetic field sensors 110a, 110b, 110c may include one or more inductive sensors (e.g., an inductive coil sensor), planar coil sensors, spin Hall sensing elements (or other Hall sensing elements), or magnetic gradiometer(s). Although TMR sensors and properties of TMR sensors may be referred to herein, it will be appreciated that any type of magnetic field sensor may be utilized, including those listed above. Magnetic field sensors 110a, 110b, 110c may have any properties of magnetic field sensors (including, e.g., TMR sensors) known in the art. For example, magnetic field sensors 110a, 110b, 110c may include a fixed layer, a tunnel layer, and a free layer. A resistance may change when the free layer is aligned with the fixed layer.

Although not shown in detail, tracking element 28 on distal portion 26 of second medical device 20 may include one or more details of tracking element 18. For example, a distal component assembly similar to distal component assembly 100, including positioning sensing system 108 and sensors 110a, 110b, and 110c, may be positioned on or within distal portion 26.

FIG. 2C is an end view of distal portion 16 of first medical device 10, and FIG. 2D is a cross-sectional view of distal portion 16 of first medical device 10 (along the plane shown in FIG. 2B), for example, looking in a distal direction from a position proximal to distalmost face 46 (FIGS. 2A and 2C). In these aspects, and as shown in FIG. 2C, distal portion 16 includes distalmost face 46. Additionally, distalmost face 46 includes working channel opening 48, lighting element 50, and camera 52. As mentioned, lighting element 50 may be formed by or otherwise coupled to optical fiber 58 (FIGS. 2A and 2B).

As shown in FIG. 2D, one or more of lighting element 50, camera 52, and pressure sensor 66 may be coupled to substrate 102. Lighting element 50 may be formed by or coupled to optical fiber 58. Additionally, substrate 102 may include a bent configuration, for example, an L-shaped configuration, for example, such that first face 102a and second face 102b are arranged approximately orthogonally to each other. As shown in FIGS. 2B and 2D, pressure sensor 66 may be positioned on substrate 102. The configuration of substrate 102 may allow for the various connections and mountings, without requiring a lateral width of distal end 16 to accommodate an entire width of substrate 102. An operator may determine a location of distal portion 16 via positioning sensing system 108. Additionally, working channel 40 (FIGS. 2D and 3A) may be free to receive one or more medical devices. In this aspect, the various distal components (lighting element 50, camera 52, optical fiber 58, pressure sensor 66, distal component assembly 100, including substrate, positioning sensing system 108 with sensors 110a, 110b, 110c, etc.) are positioned outside of working channel 40 (which may be, for example, formed by working channel lumen 56). Furthermore, in some aspects, distal end 16, for example, end cap 54 may include an outer diameter D of approximately 0.1 inches to approximately 0.2 inches, for example, approximately 0.15 inches, approximately 0.125 inches, etc.

FIGS. 3A-3E illustrate additional features of distal portion 16 of first medical device 10, including distal component assembly 100. FIG. 3A depicts distal component assembly 100 and wires 62 separated from other portions of distal portion 16 (e.g., end cap 54, working channel lumen 56, and optical fiber 58). As shown, distal component assembly 100 may be couplable and/or removable from other portions of distal portion 16. Additionally, as discussed above, camera 52 may be mounted on or otherwise coupled to substrate 102. Furthermore, distal component assembly 100 may include pressure sensor 66, for example, mounted on or otherwise coupled to a distal portion of substrate 102. For example, as shown in FIGS. 2B, 3B, and 3D, substrate 102 may include distal extension 103a, for example, to support and/or otherwise couple pressure sensor 66 to substrate 102. In these aspects, pressure sensor 66 may be electrically and/or communicable coupled to a controller (not shown) to detect and/or provide the pressure within a body lumen or body cavity to the user.

Substrate 102 may include a first face 102a (shown in plan view in FIG. 3C) and a second face 102b (oriented perpendicular to the first face and shown in plan view in FIG. 3D). First face 102a and second face 102b of substrate 102 may face radially outward when distal component assembly 100 is assembled in distal portion 16 of first medical device 10. In FIG. 3C, the first face 102a of substrate 102 faces out of the page, and the second face 102b of substrate 102 faces downward. Distal component assembly 100 may have a length A (along a longitudinal axis of distal portion 16 and first medical device 10) of approximately 0.1″ to approximately 0.3″ (e.g., approximately 0.210″). Substrate 102 may have a width B (across the first or second face, perpendicularly to the longitudinal axis) of approximately 0.09″ to approximately 0.12″ (e.g., approximately 0.101″). Furthermore, distal component assembly 100, including camera 52, may have a height H (perpendicular to length A and width B) of approximately 0.05″ to approximately 0.15″ (e.g., approximately 0.077″).

Portions of camera 52 and/or pressure sensor 66 may be mounted to substrate 102. For example, camera 52 may be mounted to first face 102a of substrate, and pressure sensor 66 may be mounted to second face 102b. As shown in FIGS. 3A-3D, distal ends of camera 52 and/or pressure sensor 66 may extend distally of distal end 104 of substrate 102. In some examples, although not shown, one or more lighting elements 50 may be mounted to a distal edge of substrate 102 (e.g., to first face 102a). Alternatively or additionally, optical fiber 58 may help to illuminate an area distal to distal portion 16 of first medical device 10. Furthermore, although not shown, optical fiber 58 and/or lighting element(s) 50 may include a transparent material, for example, at a distal portion, and the transparent material may act as a light guide to direct or re-direct light from lighting elements 50 and/or optical fiber 58 to be in front of (i.e., distal to) camera 52.

Camera 52 may be disposed approximately centrally along the width of substrate 102, for example, as shown in FIG. 3C. Lighting element 50 and/or optical fiber 58 may be positioned adjacent to (i.e., on either side of) camera 52. Additionally, camera 52 may extend distally beyond lighting element(s) 50 and/or the distal end of optical fiber 58. Camera 52 may include a camera capacitor (not shown) or any other suitable accessory components, which may be formed integrally with camera 52 or as separate element(s), for example, mounted on substrate 102.

One or more pairs (e.g., twisted pairs) of wires 62 may transmit power and/or signals to and/or from various portions of distal component assembly 100. As shown in FIG. 3C, each of wires 62 may electrically connect to a respective contact pad 116 on substrate 102 (e.g., on first face 102a) so that wires 62 are in electrical connection with respective portions of distal component assembly 100. Electrical traces or wires (not shown) may extend from each contact pad 116 to respective portions of distal component assembly 100. Including various portions of distal component assembly 100 on substrate 102 may allow fewer wires to be used than in other systems. For example, absent substrate 102, multiple wires may be required to power multiple separate lighting elements 50. Using one set of twisted wires 62, connected to substrate 102, may save space in shaft 44 and/or reduce costs as compared to using multiple wires or cables (e.g., multiple twin-ax wires) to transmit power and/or signals to and/or from respective portions of distal component assembly 100. Although shields are not depicted in FIGS. 3A-3D, one or more shields may be positioned around wires 62. Furthermore, although not shown, one or more cables may transmit power and/or signals to and from camera 52. The one or more cables may include, for example, micro-coaxial cables or other suitable cables or wires. Additionally, distal ends of each of the one or more cables may be fixed to one or more of contact pads 116 (e.g., via bonding or soldering).

Furthermore, although only one pair of wires 62 is shown, it is noted that distal component assembly 100 may include additional wires, cables, electrical and/or communications connections, etc. For example, as shown in FIGS. 3B and 3C, substrate 102 may include a plurality of contact pads 116, for example, such that each contact pad 116 may be coupled to a respective wire 62, cable, electrical and/or communications connection, etc., for example, in order to operatively couple handle portion 12 and/or umbilicus 38

As shown in FIG. 3C, substrate 102 may include thirteen contact pads 116. Contact pads 116 may be positioned on first face 102a of substrate 102, for example, in three columns of four contact pads 116, five contact pads 116, and four contact pads 116, respectively. Nevertheless, the number and/or arrangement of contact pads 116 is merely exemplary, and other numbers and/or arrangements of contact pads 116 may be utilized. For example, substrate 102 may include more or fewer contact pads 116, and one or more contact pads 116 may be arranged on second face 102b. In alternatives, the twisted pair of wires 62 may be replaced with twin-axial wires or cables (such as micro-coaxial cables) to allow soldering or bonding of conductors powering one or more portions of distal component assembly 100 with the same process as used to attach the cables (discussed above) and/or to reduce noise from electromagnetic interference.

As mentioned above, elements of position sensing system 108 may also be disposed on substrate 102. Position sensing system 108 may incorporate any of the features described in U.S. application Ser. No. 15/846,846, filed on Dec. 19, 2017, issued as U.S. Pat. No. 10,782,114, on Sep. 22, 2020, the entirety of which is incorporated herein by reference. Position sensing system 108 may include one or more magnetic field sensors 110a, 110b, 110c disposed on substrate 102. For example, as shown in FIGS. 3A, 3B, and 3D, three magnetic field sensors 110a, 110b, 110c may be disposed on second face 102b of substrate 102. Nevertheless, any alternative number of sensors may be utilized, and the three sensors 110a, 110b, 110c depicted are exemplary only.

In some examples, as shown in FIG. 3D, magnetic field sensors 110a, 110b, 110c may be arranged in a dual-axis, six-degree-of-freedom arrangement. In such an arrangement, two magnetic field sensors 110a, 110b may be oriented such that their primary sensing direction is aligned with (approximately parallel to) a longitudinal axis of first medical device 10 (which is also a longitudinal axis of component assembly 100/substrate 102). A full-Wheatstone bridge configuration may be utilized by the two magnetic field sensors 110a, 110b. The third magnetic field sensor 110c may be arranged such that its primary sensing direction is transverse (e.g., approximately orthogonal/perpendicular) to the longitudinal axis. A half-Wheatstone bridge configuration may be utilized by magnetic field sensor 110c. The Wheatstone bridges may have any characteristics of Wheatstone bridges known in the art. Sensors 110a, 110b, 110c may detect an orientation/position of distal component assembly 100 and may transmit signals indicative of the orientation/position of distal component assembly 100. A controller (not shown) may receive the signals and may calculate positioning of distal component assembly 100 using the measurements from magnetic field sensors 110a, 110b, 110c across the primary sensing direction (from magnetic field sensors 110a, 110b) and the direction orthogonal to the primary sensing direction (from magnetic field sensor 110c).

Position sensing system 108 may optionally include one or more diodes 112, for example, two diodes 112. Diodes 112 may provide high voltage protection, such as electrostatic discharge (“ESD”) protection. Diodes 112 may help to prevent damage to magnetic sensors 110a, 110b, 110c, for example, from static discharge. Diodes 112 may additionally or alternatively provide protection to aspects of camera 52. Position sensing system 108 may also optionally include a capacitor 114, for example, to help reduce noise in a voltage supplying position sensing system 108. For example, capacitor 114 may function as a decoupling capacitor, acting as a low-pass filter for any electromagnetic interference (“EMI”).

Position sensing system 108 may have other configurations within the scope of the disclosure. For example, a tri-axis configuration may be utilized for magnetic field sensors 110a, 110b, 110c, in which each of the magnetic field sensors is arranged so that its primary sensing direction is aligned with a different axis (e.g., the primary sensing directions of magnetic field sensors 110a, 110b, 110c are aligned approximately orthogonally to one another). For example, magnetic field sensor 110a may have a primary sensing direction of the X-axis. Magnetic field sensor 110b may have a primary sensing direction of the Y-axis, and magnetic field sensor 110c may have a primary sensing direction of the Z-axis. In such a tri-axis configuration, each of the magnetic field sensors 110a, 110b, 110c, may utilize a half-Wheatstone bridge configuration. Such a tri-axis configuration would require a total of eight wires—two to provide power to position sensing system 108, and two for each of the three half Wheatstone bridges of magnetic field sensors 110a, 110b, 110c. In another example, only two magnetic field sensors (e.g., magnetic field sensors 110a, 110b) may be utilized to measure six degrees of freedom, with each of magnetic field sensors 110a, 110b having a half-Wheatstone bridge configuration (or a full Wheatstone bridge configuration). In a further example, two magnetic field sensors (e.g., magnetic field sensors 110a, 110b) could be used to measure five degrees of freedom. In such an example, position sensing system 108 may be unable to measure roll. In an additional example, a single magnetic field sensor 110a could use a half Wheatstone bridge to measure five degrees of freedom.

The above examples are merely illustrative and other configurations of magnetic field sensors may be utilized. A system that utilizes three magnetic field sensors 110a, 110b, 110c in a dual-axis, six-degree-of-freedom arrangement, as shown in FIGS. 3A-3D may be beneficial due to an ability to measure six degrees of freedom while requiring only six wires 62. Alternative arrangements may also be used for lighting elements 50 and camera 52. As shown in FIGS. 3A-3D and described above, all of the contact (e.g., solder) pads 116 for wires 62 may be disposed on a single side (e.g., first face 102a) of substrate 102, which may allow for bonding of wires 62 to substrate 102 in a single manufacturing process without flipping over distal component assembly 100.

Distal component assembly 100 may also include components in addition to or in the alternative to the components described above. For example, although not shown, distal component assembly 100 also may include additional or alternative sources of lighting and/or additional or alternative imaging components (e.g., additional cameras). Distal component assembly 100 may also include additional types of sensors, such as moisture sensors, temperature sensors, or other types of sensors, which may be useful during a medical procedure.

Although the magnetic field sensors 110a, 110b, 110c are described above as being MR sensors (e.g., TMR sensors), other types of sensors may also be utilized on substrate 102. For example, one or more inductive sensors may be utilized. Inductive sensors may include one or more coils for measuring a magnetic field and determining a positioning and/or orientation of distal tip 44. Any suitable arrangement of inductive sensors may be utilized to measure a desired number of degrees of freedom. For example, inductive sensors may be positioned at least 11 degrees askew from one another (e.g., may be angled relative to a longitudinal axis of distal tip 44).

In some examples, TMR sensors may have lower costs and/or smaller sizes than inductive sensors. An inductive sensor array may require two inductive sensors (which may each be greater than 0.25″ long) placed askew from one another, to create an array measuring approximately 0.06-0.08″ (e.g., approximately 0.071″) by approximately 0.25-0.26″ (e.g., approximately 0.255″). A TMR die may have dimensions such as approximately 0.015-0.03″ (e.g., approximately 0.024″) by approximately 0.01-0.025″ (e.g., approximately 0.018″). An array of elements, such as position sensing system 108, may occupy a footprint of approximately 0.01-0.03″ (e.g., approximately 0.018″) by approximately 0.1-0.2″ (e.g., approximately 0.156″) if placed in a linear configuration. Because a TMR die and associated elements (e.g., diodes and capacitors) are modular, they may be arranged in various patterns, to, for example, minimize space and/or accommodate other features of distal component assembly 100. For example, as shown in FIG. 2A, magnetic field sensors 110a, 110b, 110c, and diodes 112 may be arranged approximately linearly. For example, as shown in FIG. 3B, diodes 112 may be proximal of magnetic field sensors 110a, 110b, 110c. An arrangement of components of distal component assembly 100 may be chosen, for example, so as to save space and provide a small footprint and size of substrate 102. Capacitor 114 may be positioned adjacent one of magnetic field sensors 110a, 110b, 110c, for example, adjacent to third magnetic field sensor 110c. Although TMR sensors may be utilized for distal component assembly 100, the disclosure is not limited to TMR sensors and may include any type of sensor, including those listed above.

To assemble distal component assembly 100, substrate 102 (e.g., multi-layered PCB) may be assembled with magnetic field sensors 110a, 110b, 110c, diodes 112, and capacitor 114 using, for example, a pick-and-place machine. Once in place, magnetic field sensors 110a, 110b, 110c, diodes 112, and capacitor 114 may be wire-bonded to substrate 102 and electrically tested. Thereafter, camera 52 and lighting element(s) 50 may be held in position using fixturing, and may have their electrical contacts bonded (e.g., at a 90-degree angle) to solder pads of substrate 102. This process of soldering camera 52 and lighting element(s) 50 may be performed on both sides of substrate 102. Finally, wires 62, any cables, etc. may be bonded on appropriate sides of substrate 102, via respective contact pads 116. For example, wires 62 for connecting to lighting element(s) 50 and different wires (not shown), for example, for connecting to position sensing system 108 may be bonded to the first side of substrate 102 in a single step. The above steps and order are merely exemplary. Wires 62, any cables, etc. may be formed into a single bundle for passing through shaft 44. Additional or alternative steps may be utilized, and different orders of steps may be performed.

Moreover, as mentioned above, although not shown, second medical device 20 may include a distal component assembly similar to distal component assembly 100. In this aspect, second medical device 20 may include a position sensing system similar to position sensing system 108, for example, in communication with one or more controllers, user interfaces, etc.

To assemble distal component assembly 100 and distal portion 16 (e.g., end cap 54), a distal end 104 of distal component assembly 100 may be inserted into a proximal opening of end cap 54. An epoxy adhesive may be applied to lighting element 50 and/or camera 52 to help seal the distal end of end cap 54, for example, distalmost face 46, from fluid ingress/egress and to help hold distal component assembly 100 (including substrate 102) in place. Other techniques may also be used to secure distal component assembly 100. For example, other types of adhesives, screws, pins, crimps, snap-fit, or other features may be used to secure distal component assembly 100 to distal portion 16 (e.g., end cap 54). Overmolding or similar techniques may also be used to secure distal component assembly 100 to distal portion 16 (e.g., end cap 54) or to secure elements of distal component assembly 100 to substrate 102. Overmolding may also be used to form distal portion 16 (e.g., end cap 54) around distal component assembly 100. For example, distal component assembly 100 may be loaded into an injection mold, and a material may be injected into the mold in order to form distal portion 16 (e.g., end cap 54). In this example, a portion of distal portion 16 (e.g., end cap 54) may be molded directly onto substrate 102, lighting element 50, camera 52, magnetic sensors 110a, 110b, 110c, diodes 112, and/or capacitor 114, and combined with other preformed or overmolded components to form distal portion 16 (e.g., end cap 54). One or more elements of distal portion 16, such as end cap 54, may be constructed using additive manufacturing or 3D printing.

In alternative examples, substrate 102 may include a flexible circuit board that is manufactured with substrate 102 in a flat configuration and then folded prior to insertion into distal portion 16 (e.g., end cap 54). In examples including a flexible circuit board, lighting element 50 may be positioned on a different plane of substrate 102 than camera 52. Components may be surface mounted on substrate 102 and then bent by an angle (e.g., by approximately 90-degrees) to face forwards (distally) or in any other direction. In further alternatives, instead of components (such as lighting element 50, camera 52, magnetic sensors 110a, 110b, 110c, diodes 112, or capacitor 114) being mounted on substrate 102 (as shown in FIGS. 2B, 3A-3E and described above), components may be embedded in substrate 102 according to any suitable methods.

Distal portion 16 may be further assembled by performing steps such as connecting articulation wires to one or more portions of distal portion 16 and assembling end cap 54 onto shaft 44. Wires 62 may be back-fed through shaft 44. Handle portion 12 may include connections for connecting to proximal ends of wires 62. For example, handle portion 12 may include a circuit board, such as a printed circuit board, having connections for the wires. Such connections may include six passive connections for wires 62 connected to position sensing system 108 (including magnetic field sensors 110a, 110b, 110c). Alternative numbers of connections may be utilized, as appropriate, depending on a configuration of distal component assembly 100. Umbilicus 38 may include conductors (e.g., wires) for carrying power and/or signals from distal component assembly 100. For example, umbilicus 38 may include six wires 62 in twisted pairs to route power and/or signals to and from position sensing system 108 through umbilicus 38.

Once assembled, first medical device 10 may be used to perform a medical procedure on a subject, for example, alone or with second medical device 20. For example, one or more of first medical device 10 and second medical device 20 may be inserted into a body lumen or body cavity of a subject. As discussed below, during the procedure, an external device may be used to generate a magnetic field near the subject. For example, the external device may be positioned on a table or other surface near the subject (e.g., near the part of the body where the body lumen is located). During the procedure, position sensing system 108 (including magnetic field sensors 110a, 110b, 110c) may transmit signals through shaft 44, to handle portion 12, and through umbilicus 38 to a controller.

The signals from position sensing system 108 may indicate a position and/or orientation of distal portion 16 within the body. Such position and/or orientation information may be fused with imaging (e.g., 2D and/or 3D imaging) performed before or during the procedure. Information from position sensing system 108 may provide an operator with information about anatomy near first medical device 10, which camera 52 alone may be unable to visualize (including anatomy outside of the body lumen in which device 10 is disposed). Furthermore, pre-procedure images may be used to automatically segment a part of the anatomy so as to provide a map (e.g., a 3D map, 3D model, or mesh) to help track or help guide first medical device 10 in real time. Such real-time tracking or guidance may decrease the amount of time, skill, and/or effort required to reach a target anatomy. In the absence of pre-procedure images, EM sensors may enable software to track a position and/or orientation of first medical device 10 and movements of first medical device 10 in order to generate a map (e.g., a 3D map) in real time, during the procedure. The generated map may guide first medical device 10 (and any EM-enabled accessories) through the subject's anatomy. As discussed below, second medical device 20 may also include a position sensing system similar to position sensing system 108, such that the position, orientation, and/or movements of second medical device 20 may also be tracked.

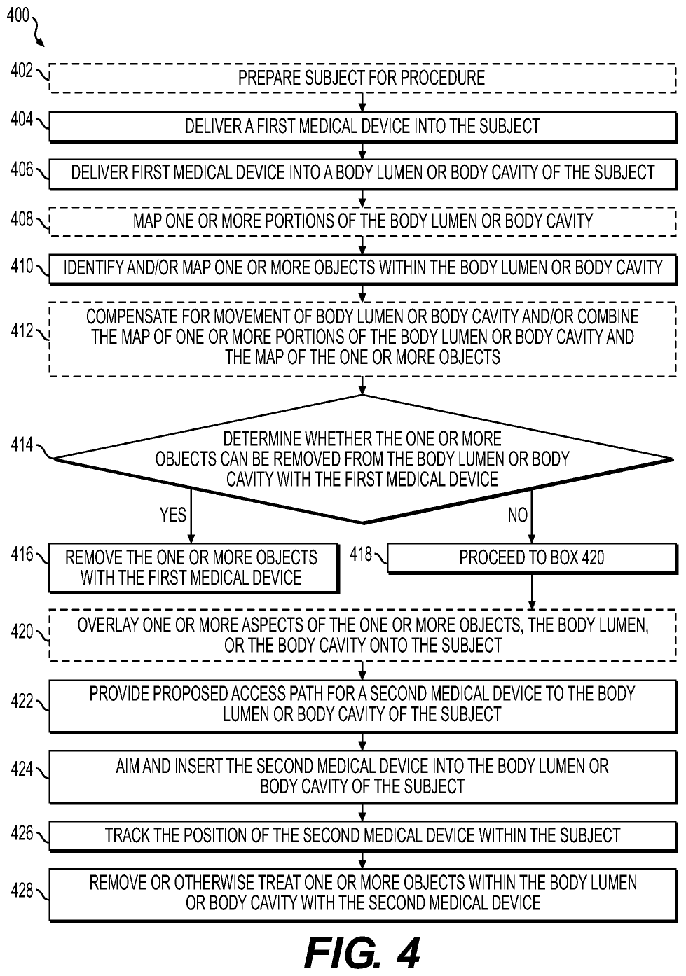

FIG. 4 illustrates an exemplary method or process 400 that one or more operators may perform with any of the medical systems, devices, or portions of systems discussed herein, for example, medical system 10. Method 400 may be performed to locate, break-up, remove, or otherwise treat one or more objects, tissues, etc. within a body lumen or a body cavity of a subject, for example, to locate, break-up, remove, or otherwise treat a kidney stone within a urethra, ureter, kidney (i.e., within a calyx) of a subject.

In some aspects, method 400 may include an initial step 402, which includes preparing the subject for a procedure. For example, step 402 may include applying a tracking patch to the skin of the subject. The tracking patch may be an electromagnetic sensor in an adhesive patch. The electromagnetic sensor may sense a position of one or more medical devices (i.e., first medical device 10 and/or second medical device 20) in a plurality of degrees of freedom. The tracking patch may be positioned on a location of the subject that is proximate to a treatment site. For example, if the treatment site is the subject's kidney, then the tracking patch may be positioned on the subject's abdomen. The tracking patch may be help track movement of the subject or a portion of the subject (i.e., the subject's chest moving as the subject inhales and/or exhales) during the procedure.

In some optional aspects, step 402 may also include additional or alternative procedures. For example, step 402 may include positioning one or more optical, fluoro-opaque, or magnetic resonance imaging (“MRI”) targets on or within the subject. In some aspects, step 402 may include delivering a contrast agent (i.e., for fluoroscopy), performing one or more scans (i.e., an MRI scan, a computerized tomography (“CT”) scan, etc.), or one or more other steps to perform one or more initial mapping and/or imaging steps. Furthermore, in some aspects, step 402 may include setting up one or more of an augmented reality headset (i.e., for the user to wear and use during the procedure), an augmented reality projector (i.e., to project one or more images on or adjacent to the subject), or other augmented reality elements.

In some aspects, step 402 may include mapping a bodily cavity or lumen with a tracked ultrasound. For example, an ultrasound transducer array may be handheld by the user and/or robotically controlled, for example, to perform a two-dimensional or three-dimensional ultrasound. In these aspects, step 402 may include mapping the body lumen or body cavity (i.e., a kidney) with ultrasound. In some aspects, one or more other steps of method 400 may also include ultrasound, for example, to track the position of first medical device 10 (i.e., distal portion 16) and/or the position of second medical device 20 (i.e., distal portion 26).

In some aspects, step 402 may include manually, automatically, or semi-automatically planning an access path to one or more objects in the body lumen or the body cavity. In this aspect, the user may manually plan one or more access paths to multiple kidney stones, for example, within multiple calyces. The user may also mark one or more structures (e.g., ribs, blood vessels, organs, etc.) to be avoided, for example, when delivering one of first medical device 10 or second medical device 20 to the body lumen or the body cavity. In some aspects, software, one or more controllers, one or more control units, or other software in handle 12 or another display may automatically identify one or more access paths and/or one or more structures to be avoided, and correspondingly display the one or more access paths and/or the one or more structures to be avoided to the user.

Method 400 includes a step 404, which includes delivering (i.e., inserting) first medical device 10 into the subject. Step 404 may include inserting first medical device 10 through a natural orifice, for example, a urethra, or may be formed by an operator, for example, via an incision. For example, step 404 may include delivering insertion portion 14 of first medical device 10 distally through a subject's urethra and bladder toward the subject's ureter(s) and kidney(s). Step 404 may include delivering first medical device 10 using an insertion sheath, a guide wire, etc.

Next, method 400 includes a step 406, which includes delivering (i.e., navigating) first medical device 10 into a body lumen or body cavity of the subject. For example, insertion portion 14 of first medical device 10 may be advanced distally through and/or to the body lumen or body cavity. Insertion portion 14 of first medical device 10 may be advanced distally manually (i.e., by the user), robotically, or semi-robotically. For example, step 406 may include distally advancing insertion portion 14 of first medical device 10 through the subject's urethra and bladder and into the subject's ureter and/or kidney(s). Step 406 may include an insertion sheath, a guide wire, etc. Alternatively or additionally, step 406 may include one or more user-controlled maneuvers, for example, using lever 30 on handle portion 12 to control the movement (i.e., articulation or deflection) of distal portion 16 of first medical device 10.

Additionally, an optional step 408 includes mapping one or more portions of the body lumen or body cavity. For example, camera 52 may be activated during step 406. Optical fiber 58 and/or illumination element 50 may also be activated during step 406, for example, to illuminate the area distal to distal portion 16 and thus in the field of view of camera 52. Furthermore, one or more aspects of position sensing system 108 may be activated during step 406. Additionally, a control unit or controller (not shown), either integral with first medical device 10 or separate from first medical device 10 may analyze one or more image and/or video signals, for example, along with a sensed position (via position sensing system 108) of distal portion 16, to create a map (e.g., a three-dimensional map) of one or more body lumens or body cavities within the subject. The map may be created based on the obtained image and/or video signals. Alternatively or additionally, the map may be created based on the sensed position of distal portion 16 and/or with sensed contact with the body lumens or body cavities within the subject. For example, if the distal portion 16 is delivered through the subject's urethra, bladder, and ureter to a kidney, optional step 408 may help to create a map of one or more of the subject's ureters and kidneys, for example, including one or more calyces.

Optional step 408 may include surveying and/or mapping the subject's bladder. For example, the control unit or controller may include stored software (i.e., in a memory), and the software may automatically create a map (i.e., a three-dimensional map) of the bladder. The map of the bladder may be created based on the image and/or video signals from camera 52, for example, in a non-contact manner. In some aspects, the map may also be based on position and/or orientation information from position sensing system 108. The map of the subject's bladder may be displayed to the user, for example, on a graphical user interface or display, which may help the user determine a spatial orientation of distal portion 16. In some aspects, the map of the bladder that may be created by software (i.e., a stored algorithm) may include a three-dimensional point cloud, for example, of surface points or a surface mesh of the bladder (i.e., with or without texture of the bladder surface). In some aspects, the map of the bladder may include an image mosaic, for example, formed by overlaying and/or otherwise combining image signal(s) and/or video signal(s) from camera 52. In some aspects, the map of the bladder may be updated, for example, constantly in real-time (i.e., more than one update per second) or otherwise periodically.

Optional step 408 may include surveying, identifying, and/or mapping one or more ureters. For example, the control unit or controller may include stored software (i.e., in a memory), and the software may automatically create a map (i.e., a three-dimensional map) of the ureter. The map of the ureter may be created based on the image signal(s) and/or video signal(s) from camera 52, for example, in a non-contact manner. In some aspects, the map may also be based on position and/or orientation information from position sensing system 108. The map of the subject's ureter may be displayed to the user, for example, on a graphical user interface or display, which may help the user determine a spatial orientation of distal portion 16.

In some aspects, the map of the ureter that may be created by software (i.e., a stored algorithm) may include a three-dimensional point cloud, for example, of surface points or a surface mesh of the ureter (i.e., with or without texture of the ureter surface). In some aspects, the map of the ureter may include an image mosaic, for example, formed by overlaying and/or otherwise combining images and/or video signals from camera 52. The map of the ureter may be based on an assumed cylindrical or tubular shape. In some aspects, the map of the ureter may be updated, for example, constantly in real-time (i.e., more than one update per second) or otherwise periodically. In some aspects, the map of the bladder and/or ureter(s) may be merged with one or more other imaging modalities, for example, images obtained from a CT scan, an MRI scan, fluoroscopy, etc. Furthermore, the map of the bladder and/or ureter(s) may help software and/or the user to delineate between bladder and one or more ureters, for example, when distal portion 16 enters a ureter from the bladder. Alternatively, one or more of the ureters may be identified manually, for example, based on the user recognizing one or more ureters in the image signal(s) and/or video signal(s) from camera 52. The user may provide one or more inputs to the controller to reflect the identification, for example, via the user interface or display (i.e., a touch screen user interface).

Next, a step 410 includes identifying and/or mapping one or more objects within the body lumen or body cavity. In this step, distal portion 16 may be positioned within or adjacent to the body lumen or body cavity, for example, within one or more portions of the subject's kidney(s). As discussed above, camera 52 may be activated during step 410. Optical fiber 58 and/or illumination element 50 may also be activated during step 410, for example, to illuminate the area distal to distal portion 16 and thus in the field of view of camera 52. Furthermore, one or more aspects of position sensing system 108 may be activated during step 410. In this aspect, distal portion 16 may be manipulated (i.e., manually or robotically) to traverse at least a portion of the body lumen or body cavity.

Additionally, image and/or video signals from camera 52, along with position signals from positioning sensing system 108, may be used to locate one or more objects within the body lumen or body cavity. For example, distal portion 16 may traverse and/or survey one or more calyces or other portions of the subject's kidney (i.e., either manually, robotically, or semi-robotically). The control unit or controller may include software that automatically recognizes one or more portions of the body lumen or body cavity and/or automatically recognizes when distal portion 16 enters or is adjacent to one or more portions of the body lumen or body cavity (e.g., recognizing one or more calyces in a kidney) based on the image signal(s) and/or video signal(s) from camera 52. The control unit or controller may alert the user when recognizing and/or entering one or more portions of the body lumen or body cavity.

Furthermore, one or more transition points (i.e., between different portions of the body lumen or body cavity, such as, from a renal pelvis to a calyx) may be used as one or more landmarks, for example, for registration and/or combination with one or more other imaging modalities (i.e., a CT scan, an MRI scan, an ultrasound scan, fluoroscopy, etc.). The image signal(s) and/or video signal(s) from camera 52, along with position signals from positioning sensing system 108, may be used to locate one or more stones within the subject's kidney, for example, within one or more calyces. Furthermore, the position of the one or more stones may be charted in a map (i.e., a three-dimensional map), which may be displayed to the user, for example, on a graphical user interface or display, which may help the user determine a spatial orientation of distal portion 16. As discussed above, the map of the subject's kidney may be displayed in real-time or periodically updated, and may include one or more of the features discussed above with respect to the bladder map and the ureter map. In addition, step 410 may include estimating the size, shape, density, etc. of the one or more objects (e.g., stones).

Next, an optional step 412 may include compensating for movement of the body lumen or the body cavity and/or combining the map of one or more portions of the body lumen or body cavity and the map of the one or more objects. For example, the map of the subject's kidney and the one or more stones may be compensated (i.e., in real-time or periodically) based on movement of the subject. In this aspect, as the subject inhales and exhales, the position and/or orientation of the subject's kidney and the one or more stones may change. In this optional step 412, information from one or more tracking patches positioned on the subject, for example, as discussed with respect to optional step 402, may be used as a dynamic reference, for example, to automatically compensate and/or adjust the map(s) of one or more portions of the body lumen or body cavity and/or the identified position(s) of the one or more objects based on movement of the one or more tracking patches. For example, in some aspects, software, one or more controllers, one or more control units, or other software in handle 12 or another display may automatically compensate and/or adjust the map(s) and/or the identified position(s).

Additionally, optional step 412 may include registering, fusing, or otherwise combining images and/or video obtained from first medical device 10 with one or more pre-operative images or other pre-operative information, for example, obtained during optional step 402. Step 412 may include one or more rigid and/or elastic approaches. The registered, fused, or otherwise combined images and/or video may be displayed to the user, for example, via a graphical user interface or other display (i.e., as two-dimensional and/or three-dimensional images or videos). In this aspect, the graphical user interface or other display may indicate information in one or more ways, for example, as a multiplanar reformation (MPR) display, a needle aligned images, a full scene image, etc.

Next, a step 414 includes determining whether the one or more objects can be removed from the body lumen or body cavity with the first medical device. For example, as discussed above, step 410 may include determining the size and/or shape of the one or more objects (e.g., one or more stones in the subject's kidney). Step 414 may include determining whether the one or more objects are smaller or of an appropriate size or shape to be removed with first medical device 10, for example, through working channel 40 (FIG. 3A). Step 414 may include a video and/or image-based analysis by the controller, for example, to determine one or more of a size, shape, dimensions, center of mass, etc. of the one or more objects. Alternatively or additionally, step 414 may include determining a classification and/or type of the one or more objects (i.e., the type of kidney stone). Any of these aspects of the one or more objects may be displayed to the user, for example, on a user interface or other display. If the one or more objects can be removed with first medical device 10, then method 400 proceeds to step 416. However, if the one or more objects cannot be removed with first medical device, then step 418 includes continuing method 400, for example, proceeding to step 420.

Step 416 includes removing the one or more objects with first medical device 10. For example, suction may be applied through working channel 40 (i.e., from a vacuum source and controlled by, for example, suction valve 34 on handle portion 12), such that one or more objects (i.e., stones) may be drawn toward working channel opening 48 of first medical device 10. In some aspects, the one or more objects may be removed proximally, for example, through working channel 40. Alternatively or additionally, one or more objects may be removed proximally by withdrawing first medical device 10 from the subject. In other aspects, one or more retrieval devices (e.g., baskets, graspers, or other retrieval devices) may be delivered to the body lumen or body cavity, for example, through port 32, through working channel 40, and out of working channel opening 48. The one or more retrieval devices may be manipulated (i.e., moved, expanded, contracted, etc.) to capture the one or more objects. The one or more retrieval devices may then be removed, for example, retracted proximally, to remove the one or more objects from the body lumen or body cavity.

Furthermore, alternatively or additionally, an energy delivery device (i.e., a laser fiber) may be delivered to the body lumen or body cavity (i.e., through working channel 40). Then, energy (i.e., laser energy) may be delivered toward the one or more objects to break up the one or more objects. In some aspects, the broken-up objects may then be removed from the body lumen or body cavity (e.g., via suction, one or more retrieval devices, etc.). Throughout the removal steps, the position of distal portion 16 may be tracked via position sensing system 108. In some aspects, the map of the body lumen or body cavity (including the one or more objects) may be continuously or periodically updated, which may help track the position of the distal portion 16 and the position and/or removal of the one or more objects.

As mentioned, if the one or more objects cannot be removed from the body lumen or body cavity with first medical device 10, then method 400 may proceed to optional step 420. Optional step 420 overlaying one or more aspects of the one or more objects, the body lumen, or the body cavity onto the subject. For example, optional step 420 may include overlaying one or more aspects of the one or more objects, the body lumen, or the body cavity onto the subject. For example, step 420 may include projecting one or more images or maps of the body lumen or body cavity, and/or the one or more objects to be removed. In one aspect, step 420 may include projecting one or more images or maps of the kidney(s), calyces, kidney stone, an access site, etc. on the subject (i.e., onto the skin of the subject), for example, an insertion location and/or an insertion route from the exterior of the subject to the kidney or a calyx within the kidney. Step 420 may include a light projector that projects one or more images onto the subject's body (i.e., onto the skin of the subject).

Next, step 422 includes providing a proposed access path for second medical device 20 to the body lumen or body cavity of the subject. Step 422 may include one or more maps, images, etc., for example, based on the information received in one or more of steps 402, 404, 406, 408, 410, etc. Step 422 may include displaying the one or more maps, images, etc. on a display or user interface. Alternatively or additionally, step 422 may include using augmented reality to display the one or more maps, images, etc. to the user, for example, with an augmented reality headset. Alternatively or additionally, step 422 may include one or more alignment markers or other registration elements, for example, to help ensure the tracking in a system coordinate space (i.e., of the subject's body).

Next, a step 424 includes aiming and/or inserting second medical device 20 into the body lumen or body cavity of the subject. Step 424 may include making one or more incisions in the subject and/or using one or more insertion devices (i.e., guidewires, guidance sheaths, dilation sheaths, etc.). Step 424 may include delivering distal portion 26 and a portion of insertion portion 24 of second medical device 20 into the subject, for example, using the one or more insertion devices. In some aspects, step 424 may include inserting second medical device 20 through an incision in a rear or side (i.e., a flank) of the subject. Step 424 may include inserting second medical device 20 to the subject's kidney (i.e., to one or more calyces).

Furthermore, a step 426 may include tracking the position of second medical device 20 within the subject. In this aspect, tracking element 28 on distal portion 26 of second medical device 20 may be used to track the position of distal portion 26, for example, during the aiming and/or insertion of second medical device 20 into the body lumen or body cavity of the subject (i.e., during step 424). As mentioned above, tracking element 28 may include one or more position sensing systems, for example, similar to positioning sensing system 108. Tracking the position of second medical device 20 (i.e., the position of distal portion 26) in step 426 may help to ensure that second medical device 20 is effectively and efficiently delivered to the body lumen or body cavity (i.e., to the appropriate calyx of the subject's kidney).

Then, a step 428 includes removing or otherwise treating the one or more objects within the body lumen or body cavity with second medical device 20. For example, insertion portion 24 of second medical device 20 may include one or more internal lumens or working channels. As discussed above with respect to first medical device 10, step 428 may include applying suction through the lumen or working channel of second medical device 20 (i.e., from a vacuum source), such that one or more objects (i.e., stones) may be drawn toward the opening of the lumen or working channel of second medical device 20. For example, second medical device 20 may be a nephroscope. In some aspects, the one or more objects may be removed proximally, for example, through the lumen or working channel. Alternatively or additionally, one or more objects may be removed proximally by withdrawing second medical device 20 from the subject. In other aspects, one or more retrieval devices 42 (e.g., baskets, graspers, or other retrieval devices) may be delivered to the body lumen or body cavity, for example, through the lumen or working channel of second medical device 20. The one or more retrieval devices may be manipulated (i.e., moved, expanded, contracted, etc.) to capture the one or more objects, for example, by manipulation of actuation member 60. The one or more retrieval devices 42 may then be removed, for example, retracted proximally, to remove the one or more objects from the body lumen or body cavity. Furthermore, alternatively or additionally, an energy delivery device (i.e., a laser fiber) may be delivered to the body lumen or body cavity (i.e., through the lumen or working channel of second medical device 20). Then, energy (i.e., laser energy) may be delivered toward the one or more objects to break up the one or more objects. In some aspects, the broken-up objects may then be removed from the body lumen or body cavity (e.g., via suction, one or more retrieval devices, etc.).

Throughout the removal steps, the position of distal portion 26 may be tracked via a position sensing system similar to position sensing system 108. Furthermore, the map of the body lumen or body cavity (including the one or more objects) may be continuously or periodically updated, for example, via camera 52 of first medical device 10, which may help track the position of the distal portion 26 of second medical device 20 and/or the position and/or removal of the one or more objects

Additionally, method 400 may include repeating one or more of the steps. For example, if the body lumen or body cavity includes more than one object, then one or more of steps 416, 420, 422, 424, 426 or 428 may be repeated as many times as necessary to ensure that the objects are removed from the body lumen or body cavity. Furthermore, one or more of first medical device 10 and second medical device 20 may be repositioned, for example, to another portion of the body lumen or body cavity (i.e., to another calyx within the kidney of the subject) in order to update the map of the objects within the body lumen or body cavity, remove the one or more objects from the body lumen or body cavity, ensure that the one or objects have been removed from the body lumen or body cavity, etc.

As discussed above, first medical device 10 and/or second medical device 20 may be used to perform a medical procedure on a subject. For example, first medical device 10 may be inserted into a body lumen or body cavity (e.g., a urethra) of a subject, and/or second medical device 20 may be inserted into the subject (e.g., through an incision toward a kidney). During the procedure, an external device may be used to generate a magnetic field near the subject. For example, the external device may be positioned on a table or other surface near the subject (e.g., near the part of the body where the body lumen or body cavity is located).

During the procedure, position sensing system 108 (including magnetic field sensors 110a, 110b, 110c) may transmit signals through shaft 44, to handle portion 12, and through umbilicus 38 to a controller. The signals from position sensing system 108 may indicate a position and/or orientation of distal portion 16 within the body. Similarly, tracking element 28 on distal portion 26 of second medical device 20 may include a position sensing system, which may transmit one or more signals to the controller to indicate a position and/or orientation of distal portion 26 within the body. Such position and/or orientation information may be fused with imaging (e.g., 3D imaging) performed before the procedure and/or imaging (e.g., 2D imaging or video) received from camera 52 of first medical device 10.

Information from position sensing system 108 and/or the positioning sensing system of second medical device 20 may help to provide a user with information about anatomy near first medical device 10 and/or second medical device 20, which camera 52 alone may be unable to visualize (including anatomy outside of the body lumen in which first medical device 10 and/or second medical device 20 is disposed). For example, positioning sensing system 108 and/or the positioning sensing system of second medical device 20 may provide information on the location of first medical device 10 and/or second medical device 20 with respect to one or more reference locations. In some aspects, information from positioning sensing system 108 and/or the positioning sensing system of second medical device 20 may be registered with one or more pre-operative images, for example, as discussed below. Furthermore, information from positioning sensing system 108 and/or the positioning sensing system of second medical device 20 may provide anatomical context to the user, for example, information on the location, position, and/or orientation of first medical device 10 and/or second medical device 20. Additionally, if a 3D map is available (e.g., via a pre-operative or intra-operative imaging procedure), the location, position, and/or orientation of first medical device 10 and/or second medical device 20 may be displayed relative to the 3D map, for example, facilitating guidance of first medical device 10 and/or second medical device 20 during the procedure.

Furthermore, pre-procedure images may be used to automatically segment a mesh of the anatomy so as to provide a map (e.g., a 3D map) to track first medical device 10 and/or second medical device 20 in real time. Such real-time tracking may decrease the amount of time, skill, and/or effort required to reach a target anatomy. In the absence of pre-procedure images, EM sensors may enable software to track a position of first medical device 10 and/or second medical device 20 and movements of first medical device 10 and/or second medical device 20 in order to generate a map (e.g., a 3D map) in real time, during the procedure. The generated map may guide first medical device 10 and/or second medical device 20 (and any EM-enabled accessories) through the subject's anatomy.

In an alternative, distal portion 16 of first medical device 10 and/or of distal portion 26 of second medical device 20 may include elements to generate a magnetic field, and an external device may include elements that measure the magnetic field and determine positioning and/or orientation of distal portion 16 or distal portion 26. For example, distal portion 16 of first medical device 10 and/or of distal portion 26 of second medical device 20 may include one or more coils (e.g., solenoids). Circuitry element(s), such as wire(s), circuit board(s), and/or one or more other component(s) mounted on circuit board(s) may transmit current through the coil(s). The coil(s) may thus generate a magnetic field. The external device may include one or more sensors (including, for example, any of the types of sensors described above) or assemblies for measuring the magnetic field emitted by the coil(s). Measurements from the sensors or assemblies of the external device may be used (e.g., by a controller) to determine a position of distal portion 16 of first medical device 10 and/or of distal portion 26 of second medical device 20.

Any methods or portions of methods described in this disclosure may be performed by one or more processors of a computer system. The one or more processors may be configured to perform such methods by having access to instructions (e.g., software or computer-readable code) that, when executed by the one or more processors, configure and/or cause the one or more processors to perform the methods. Such instructions may be stored in a memory of the computer system.

Instructions executable by one or more processors may also be stored on a non-transitory computer-readable medium. Therefore, whenever a computer-implemented method is described in this disclosure, this disclosure shall also be understood as describing a non-transitory computer-readable medium storing instructions that, when executed by one or more processors of a computer system, configure and/or cause the one or more processors to perform the computer-implemented method. Examples of non-transitory computer-readable media include RAM, ROM, solid-state storage media (e.g., solid-state drives), optical storage media (e.g., optical discs), and magnetic storage media (e.g., hard disk drives). A non-transitory computer-readable medium may be part of the memory of a computer system or separate from any computer system.

A computer system may include one or more computing devices. If a computer system includes a plurality of processors, the plurality of processors may be included in a single computing device or distributed among a plurality of computing devices. A processor may be a central processing unit (CPU), a graphics processing unit (GPU), or another type of processing unit. The term “computational device,” as used in this disclosure, is interchangeable with “computing device.” An “electronic storage device” may include any of the non-transitory computer-readable media described above.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. A medical system, comprising:
a first medical device, wherein the first medical device includes a handle and an insertion portion with a first working channel, wherein a distal portion of the insertion portion of the first medical device includes a first tracking element and at least one camera; and
a second medical device, wherein the second medical device includes a handle and an insertion portion, wherein a distal portion of the insertion portion of the second medical device includes a second tracking element,
wherein the first tracking element is configured to provide a signal that indicates a position or orientation of the distal portion of the first medical device, and wherein the second tracking element is configured to provide a signal that indicates a position or orientation of the distal portion of the second medical device.

2. The medical system of claim 1, wherein the distal portion of the first medical device further includes one or more lighting elements.

3. The medical system of claim 2, wherein the first tracking element, the at least one camera, and the one or more lighting elements are coupled to a substrate within the distal portion of the first medical device.

4. The medical system of claim 3, wherein the substrate includes a first face and a second face, wherein the first face and the second face are arranged approximately orthogonally to each other within the distal portion of the first medical device, and wherein a proximal portion of the substrate includes a plurality of contact pads.

5. The medical system of claim 3, wherein the first tracking element includes a position sensing system that includes one or more position or orientation sensors.

6. The medical system of claim 5, wherein the position sensing system includes a first magneto-resistive sensor, a second magneto-resistive sensor, and a third magneto-resistive sensor, wherein the first magneto-resistive sensor, the second magneto-resistive sensor, and the third magneto-resistive sensor are coupled to the substrate.

7. The medical system of claim 6, wherein the first magneto-resistive sensor has a first primary sensing direction, wherein the second magneto-resistive sensor has a second primary sensing direction, wherein the third magneto-resistive sensor has a third primary sensing direction, wherein the first magneto-resistive sensor and the second magneto-resistive sensor are arranged so that each of the first primary sensing direction and the second primary sensing direction is approximately parallel with a longitudinal axis of the substrate.

8. The medical system of claim 7, wherein the third magneto-resistive sensor is arranged such that the third primary sensing direction is transverse to the longitudinal axis of the substrate.

9. The medical system of claim 8, wherein the position sensing system is configured to measure at least five degrees of freedom.

10. The medical system of claim 6, wherein the distal portion of the first medical device further includes one or more diodes and one or more capacitors, wherein the one or more diodes and the one or more capacitors are coupled to the substrate.

11. The medical system of claim 1, wherein the handle of the first medical device includes a lever configured to control an articulation or deflection of the distal portion of the first medical device, wherein the handle of the first medical device includes a port connected to the first working channel, and wherein the handle of the first medical device includes a suction valve to control suction applied through the first working channel.

12. The medical system of claim 1, wherein the first medical device includes an optical fiber extending from the handle portion to the distal portion of the insertion portion of the first medical device, wherein a distal end of the optical fiber is adjacent to the at least one camera, and wherein the first medical device further includes a pressure sensor positioned at the distal portion of the first medical device.

13. The medical system of claim 1, wherein the second medical device is a nephroscope, and wherein the nephroscope includes a working channel.

14. The medical system of claim 13, further comprising a retrieval device configured to be delivered through the working channel of the nephroscope.

15. The medical system of claim 1, wherein the first medical device includes an end cap at the distal portion, and wherein the end cap at least partially surrounds the first tracking element.

16. A medical device, comprising:
a handle, including a port; and
an insertion portion with a working channel connected to the port, wherein a distal portion of the insertion portion includes a tracking element and at least one camera,
wherein the tracking element include a plurality of magneto-resistive sensors, and wherein the tracking element is configured to provide a signal that indicates a position or orientation of the distal portion of the medical device, and
wherein the tracking element and the at least one camera are mounted on a substrate within a distal portion of the insertion portion.

17. The medical device of claim 16, wherein the substrate includes a first face and a second face, wherein the first face and the second face are arranged approximately orthogonally to each other within the distal portion of the insertion portion of the medical device, wherein the at least one camera is mounted on the first face of the substrate, and wherein the plurality of magneto-resistive sensors includes a first magneto-resistive sensor, a second magneto-resistive sensor, and a third magneto-resistive sensor, wherein the first magneto-resistive sensor, the second magneto-resistive sensor, and the third magneto-resistive sensor are coupled to the second face of the substrate.

18. The medical device of claim 17, wherein the handle further comprises an image capture button, a lever, a port, and a suction valve;
wherein the image capture button is configured to control activation of the camera at the distal portion of the insertion portion;
wherein the lever is configured to control an articulation or deflection of the distal portion of the medical device; and
wherein the suction valve is configured to control suction applied through the working channel.

19. A method, comprising:
delivering a first medical device into a subject, wherein the first medical device includes a handle and an insertion portion, wherein the insertion portion includes a working channel, wherein a distal portion of the insertion portion includes a camera and a tracking element, and wherein the tracking element includes one or more magneto-resistive sensors;
delivering the distal portion of the insertion portion the first medical device into a body lumen or body cavity of the subject;
identifying and/or mapping one or more objects within the body lumen or the body cavity with the camera;
determining whether the one or more objects can be removed from the body lumen or body cavity with the working channel of the first medical device;
upon determining that the one or more objects cannot be removed with the working channel of the first medical device, providing a proposed access path for a second medical device to the body lumen or body cavity of the subject;
aiming and inserting the second medical device to the body lumen or the body cavity of the subject;
tracking a position of the second medical device within the subject; and
removing or otherwise treating the one or more objects within the body lumen or body cavity with the second medical device.

20. The method of claim 19, further comprising:
an initial step of preparing the subject for a procedure by performing one or more of an ultrasound, a CT scan, or an MRI scan of a kidney of the subject; and
before providing the access path for the second medical device, overlaying one or more overlaying one or more aspects of the kidney or one or more kidney stones on the subject.
